(19) United States
(12) Patent Application Publication
(10) Pub. No.: US 2018/0322955 A1
(43) Pub. Date: Nov. 8, 2018

(54) VISUALLY INDICATING CONTRIBUTIONS OF CLINICAL RISK FACTORS

(71) Applicant: KONINKLIJKE PHILIPS N.V., Eindhoven (NL)

(72) Inventors: Merlijn Sevenster, Haarlem (NL); Thomas Andre Forsberg, Hayward, CA (US)

(21) Appl. No.: 15/968,782

(22) Filed: May 2, 2018

Related U.S. Application Data
(60) Provisional application No. 62/500,696, filed on May 3, 2017.

(51) Int. Cl. G16H 50/30; G16H 50/20; G06N 99/00

(52) U.S. Cl. CPC G16H 50/30; G16H 10/60; G06N 99/005; G16H 50/20

(57) ABSTRACT

The present disclosure relates to visually indicating contributions of clinical risk factors to various model-based health assessments. In various embodiments, a plurality of clinical risk factors associated with a patient may be received and applied as input across a trained model to generate a score associated with the patient. Based on the trained model, first and second contributions of respective first and second clinical risk factors of the plurality of clinical risk factors to the score may be determined. A graphical user interface may be provided on a display, and the graphical user interface may include at least a first visual indication of the first contribution and a second visual indication of the second contribution.

### CROSS-REFERENCE TO RELATED APPLICATION

The present application claims the benefit of U.S. Provisional Application No. 62/500,696, filed May 3, 2017. These applications are hereby incorporated by reference herein.

### TECHNICAL FIELD

The present disclosure is directed generally to health care. More particularly, but not exclusively, various inventive methods and apparatus disclosed herein relate to visually indicating contributions of clinical risk factors to various model-based health assessments.

### BACKGROUND

Health care providers such as clinicians often lack sufficient insight into the relative contributions of individual clinical risk factors to various health assessments (e.g., predictions, adverse events, etc.). Consequently, they are less able to make informed and/or optimal clinical decisions. Clinical risk factors are researched for a variety of purposes, such as accurately predicting risks to patients resulting from various medical interventions. In some instances, clinical risk factors are used in conjunction with (e.g., as input for) trained models, such as logistic regression models, neural networks, etc., to generate output that is indicative of, for instance, likelihood of an adverse outcome following a medical intervention. For example, logistic regression models may weigh a contribution of each clinical risk factor to determine the likelihood of an adverse outcome.

As one non-limiting example, an Acute Kidney Injury (“AKI”) model may receive the following clinical risk factors as inputs: hypotension (5); chronic heart failure (5); age (e.g., <75 years→44); anemia (3); diabetes (3); and estimated glomerular filtration rate (“eGFR”) <60 ml/min/1.73 m² (2, 4, or 6). Clinical studies have shown that these clinical risk factors contribute to output of the AKI model in accordance with the relative weights indicated above in parenthesis. However, clinicians may not be made aware of the relative contribution of each clinical risk factor to output of the AKI model. At best, some existing user interfaces indicate whether or not each clinical risk factor contributes to the assessment, e.g., by coloring an indication of the risk factor as red (indicating that the clinical risk factor contributed) or black (no contribution). But these interfaces do not convey a relative contribution of each clinical risk factor.

### SUMMARY

The present disclosure is directed to methods and apparatus for visually indicating contributions of clinical risk factors to various model-based health assessments. In various embodiments, a plurality of clinical risk factors associated with a particular patient may be received, e.g., at one or more input interfaces of a computing device and/or from one or more databases. These clinical risk factors may come in many forms, including but not limited to measured vital signs (heart rate, temperature, blood pressure, etc.), test results (e.g., blood creatinine test, eEFG), chronic conditions (e.g., diabetes, heart disease, hypertension, hypotension, arthritis, asthma, cancer, osteoporosis, cystic fibrosis, alzheimer's, etc.), general factors (e.g., gender, age, oral health, obesity, etc.), behavioral factors (e.g., exercise history/habits, tobacco use, alcohol consumption, drug use, diet, dental practices, etc.), and so forth.

In some implementations, the received clinical risk factors may be applied as input to one or more algorithms, such as across a trained model, to generate output such as a classification or score associated with the patient. Such output can be indicative of a wide variety of conclusions, such as likelihood of various adverse outcomes resulting from various medical interventions, likelihood of developing various diseases and conditions, life expectancy, patient acuity, and so forth. As noted above, without more information, such model-based techniques may be akin to “black boxes” which may provide the clinician with conclusory information, without providing sufficient details about relative contribution of various clinical risk factors.

Accordingly, in various embodiments, relative contributions of various clinical risk factors to the score may be determined. In some embodiments, these relative contributions may be determined based on the model used to generate the output (or conclusion). For example, with logistic regression models, various techniques exist to determine how much each individual input contributed to a final score, such as examining weights associated with various inputs. With neural networks, weights associated with neurons may be considered, and in some cases, neurons at various levels of the neural network, such as so-called “top level” neurons (e.g., the last level of neurons that contribute to an ultimate output of the neural network) may be considered.

Once the relative contributions of at least some input clinical risk factors are known, in various embodiments, visual indications may be provided, e.g., on a display, that convey the relative contributions of the clinical risk factors. For example, in some embodiments, an array or matrix of blocks (or bars, or “tiles”) that correspond to clinical risk factors may be rendered with spatial dimensions, such as heights, that correspond to a relative contribution of the underlying clinical risk factors. In some embodiments, blocks may be rendered three-dimensionally, e.g., so that the blocks extend along a simulated z-axis (e.g., towards a viewer of the display) by heights that correspond to contributions of their underlying clinical risk factors. Additionally or alternatively, other visual techniques may be used to convey relative contributions of clinical risk factors to model-based assessments, such as font sizes, font colors, colors generally (e.g., a heat map), and so forth.

Generally, in one aspect, a method may include: receiving a plurality of clinical risk factors associated with a patient; applying the plurality of clinical risk factors as input across a trained model to generate a score associated with the patient; determining, based on the trained model, a first contribution of a first clinical risk factor of the plurality of clinical risk factors to the score; determining, based on the trained model, a second contribution of a second clinical risk factor of the plurality of clinical risk factors to the score; and providing, on a display, a graphical user interface that includes at least a first visual indication of the first contribution and a second visual indication of the second contribution.

In various embodiments, the first and second visual indications may include first and second blocks having first and second heights that are selected based on the first and second contributions, respectively. In various embodiments, the display is a two-dimensional display having an x-axis and a y-axis, and the first and second blocks may be rendered three-dimensionally so that the first and second blocks extend along a simulated z-axis by the first and second heights.

In various embodiments, the first and second contributions may be determined based on first and second weights obtained from the trained model. In various embodiments, trained model may be a logistic regression model. In various embodiments, the trained model may be a neural network. In various versions, the first and second contributions may be determined based on late level neurons of the neural network. In various embodiments, the first and second visual indications may include one or more font attributes used to display, in the graphical user interface, text associated with the first and second clinical risk factors, respectively.

It should be appreciated that all combinations of the foregoing concepts and additional concepts discussed in greater detail below (provided such concepts are not mutually inconsistent) are contemplated as being part of the inventive subject matter disclosed herein. In particular, all combinations of claimed subject matter appearing at the end of this disclosure are contemplated as being part of the inventive subject matter disclosed herein. It should also be appreciated that terminology explicitly employed herein that also may appear in any disclosure incorporated by reference should be accorded a meaning most consistent with the particular concepts disclosed herein.

### BRIEF DESCRIPTION OF THE DRAWINGS

In the drawings, like reference characters generally refer to the same parts throughout the different views. Also, the drawings are not necessarily to scale, emphasis instead generally being placed upon illustrating the principles of the disclosure.

FIG. 1 schematically illustrates an environment in which disclosed techniques may be employed, in accordance with various embodiments.

FIG. 2A demonstrates how a conventional graphical user interface may convey whether or not particular clinical risk factors contributed to a particular health assessment.

FIG. 2B demonstrates how relative contributions of a plurality of clinical risk factors to a particular health assessment may be presented, in accordance with various embodiments.

FIG. 3A schematically illustrates an example method of training a model configured with selected aspects of the present disclosure, in accordance with various embodiments.

FIG. 3B schematically illustrates an example method of applying clinical risk factors as input across a model and conveying output in accordance with the present disclosure, in accordance with various embodiments.

FIG. 4 schematically depicts components of an example computer system, in accordance with various embodiments.

### DETAILED DESCRIPTION

Clinical risk factors are researched for a variety of purposes, such as accurately predicting risks to patients resulting from various medical interventions. In some instances, clinical risk factors are used in conjunction with (e.g., as input for) trained models, such as logistic regression models, neural networks, etc., to generate output that is indicative of, for instance, likelihood of an adverse outcome following a medical intervention. For example, logistic regression models may weigh a contribution of each clinical risk factor to determine the likelihood of an adverse outcome. Health care providers such as clinicians often lack sufficient insight into the relative contributions of individual clinical risk factors to adverse events. Consequently, they are less able to make informed and/or optimal clinical decisions. Accordingly, techniques are described herein for visually indicating contributions of clinical risk factors to various model-based health assessments.

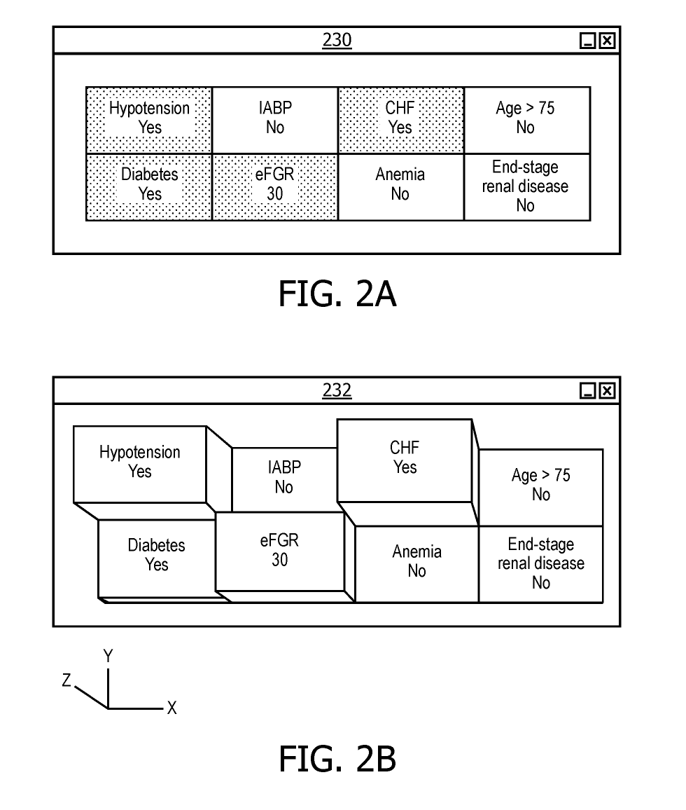

FIG. 2 depicts an example environment 100 in which various components may interoperate to perform techniques described herein. The environment 100 includes a variety of components that may be configured with selected aspects of the present disclosure, including a health assessment engine 102, one or more electronic medical record (“EMR”) databases 104, one or more model databases 106, and/or one or more miscellaneous pieces of medical equipment 108. A variety of client devices 112, such as a smart phone 112a, a laptop computer 112b, a tablet computer 112c, and a smart watch 112d, may also be in communication with other components depicted in FIG. 2. In some embodiments, the components of FIG. 2 may be communicatively coupled via one or more wireless or wired networks 114, although this is not required. And while the components are depicted in FIG. 2 separately, it should be understood that one or more components depicted in FIG. 2 may be combined in a single computer system (which may include one or more processors), and/or implemented across multiple computer systems (e.g., across multiple servers). For example, model database(s) 106 may be integral with, for instance, health assessment engine 102.

Health assessment engine 102 may be configured to apply various data points, such as clinical health risks associated with a patient, as input across one or more models stored in model database(s) 106 to generate output (e.g., classifications, scores) indicative of various medical assessments. Output generated by health assessment engine 102 may be indicative of a wide variety of medical assessments, such as medical intervention risks (e.g., probability of adverse outcomes resulting from medical intervention), medical conditions, clinical decision support (“CDS”) assessments (e.g., the aforementioned AKI, bleeding risks, mortality, etc.), and so forth.

Health assessment engine 102 may utilize a variety of different types of models from model database 106. In some embodiments, health assessment engine 102 may utilize various types of models trained (unsupervised or supervised) to generate output indicative of various assessments, such trained logistic regression models, linear regression models, decision trees, random forests, artificial neural networks, support vector machines, and so forth.

Electronic medical record (“EMR”) database 104 may include records of observed and/or observable health information associated with a plurality of patients. For example, EMR database 104 may include a plurality of EMRs that include, among other things, data indicative of one or more clinical risk factors of the patients. Example clinical risk factors are described elsewhere herein. In other embodiments, EMR database 104 may include anonymized clinical risk factors associated with a plurality of patients, e.g., collected as part of a study. These anonymized clinical risk factors, together with known outcomes, may be used to implement supervised training of various types of models that may be contained in model database 106 and applied by health assessment engine 102.

EMR database 104 may also include, for instance, information pertaining to treatment of patients by medical personnel, include various characteristics of treatment provided to patients. For example, in addition to various vital sign measurements collected from each patient (e.g., blood pressure, pulse rate, blood sugar levels, temperature, lactose levels, etc.), EMR database 104 may include records indicative of characteristics of how the vital signs were obtained. For example, EMR database 104 may include data indicative of whether a particular vital sign measurement was taken invasively or non-invasively, how often a particular vital sign was taken/measured, a stated reason for taking the measurement, and so forth. More generally, EMR database 104 may include records indicative of characteristics of treatment provided to patients. These records may include but are not limited to whether a particular medicine or therapy was prescribed and/or administered, a frequency at which the medicine/treatment is prescribed/administered, an amount (or dosage) of medicine/treatment prescribed/administered, whether certain therapeutic and/or prophylactic steps are taken, whether, how frequently, and/or how much fluids are being administered, and so forth.

In some embodiments, one or more models in model database 106 may be trained using one or more patient feature vectors containing various clinical risk factors obtained from EMR database 104 as well as known assessments/outcomes (i.e. labels). Once a given model is sufficiently trained, health assessment engine 102 may apply, as input across the trained model, clinical risk factor feature vectors associated with subsequent patients, and may generate, as output, various health assessments. In essence, each model in model database 106 “learns” correlations or mappings between clinical risk factors of previous patients and known outcomes, and then uses that knowledge to “guess” or “estimate” one or more health assessments for a subsequent patient based on clinical risk factors associated with the subsequent patient.

Referring to FIG. 2A, an example of how a medical assessment may be presented using a “conventional” graphical user interface (“GUI”) 230 is shown. In this example, the depicted clinical risk factors pertain to an AKI assessment as described above. Each clinical risk factor is represented by a tile (e.g., a simple rectangle). Clinical risk factors that contribute to the AKI assessment are shaded in FIG. 2A. Clinical risk factors that did not contribute to the AKI assessment are not shaded in FIG. 2A. A problem with such a GUI is that there is no indication as to relative contributions of each clinical risk factor. A clinician studying GUI 230 may learn that hypotension, chronic heart failure (“CHF” in FIG. 2A), diabetes, and eGFR all contributed to the AKI assessment, but not how much each contributed, e.g., relative to the others.

Accordingly, in various embodiments, health assessment engine 102 or another component may render, or provide data that enables other computing devices to render, a GUI that more effectively conveys relative contributions of one or more clinical risk factors to a particular model-based health assessment. FIG. 2B depicts one example of how disclosed techniques may be used to provide, as output on a GUI 232, relative contributions of various clinical risk factors to a model-based health assessment, in accordance with various embodiments.

In FIG. 2B, the same health assessment—AKI in view of the same clinical risk factors—is being considered. However, instead of simply presenting the clinical risk factors visually in a binary fashion—contributed or did not contribute—the clinical risk factors are presented with some visual indication of their relative contribution to the health assessment. Standard computing device displays are two-dimensional with an x-axis and a y-axis. However, in this example, each “tile” or block is rendered three-dimensionally so that the tiles extend along a simulated z-axis (out from the page) by a height that corresponds to the underlying clinical risk factor's relative contribution to the health assessment. For example, hypotension (low blood pressure) is known to be a heavy contributor to AKI scores. If hypotension is present, then that heavily affects the AKI score relative to most other clinical risk factors. In other words, hypotension is assigned a relatively large weight (or “theta”) in an underlying model used to compute the AKI score. Thus, the tile corresponding to hypotension (top left) extends outwards along the z-axis by a relatively large amount compared to most of the other types. Chronic heart failure (“CHF”) is an even heavier contributor (i.e., has an even greater assigned weight in the underlying model), and thus the tile corresponding to chronic heart failure (top row, second from left) extends even further out from the page. A clinician viewing GUI 232 may quickly determine that chronic heart failure and hypotension are the two leading clinical risk factors that contributed to the AKI assessment.

By contrast, while the clinical risk factor eFGR=30 contributes to the AKI score, it contributes less than hypotension or chronic heart failure (e.g., either is assigned a lesser weight in the model or its relatively low value limits its contribution). Consequently, the tile corresponding to eFGR (bottom row, second from left) extends a smaller distance out of the page along the z-axis. Similarly, while diabetes is present and therefore contributes to the AKI score, its relative contribution is less than other clinical risk factors. Accordingly, the tile corresponding to diabetes (bottom left) extends only a small distance along the z-axis. The remaining clinical factors did not contribute to the AKI score (e.g., because they weren't present) and therefore do not extend along the z-axis at all.

The example GUI 232 of FIG. 2B is just one example of how relative contributes of clinical risk factors to a model-based health assessment may be visually depicted. There are numerous other ways that such relative contributions could be depicted. In some embodiments, graphical elements and/or fonts associated with clinical risk factors may be altered in accordance with, for example, a color scale (e.g., a heat map) that visually conveys relative contributions of clinical risk factors. For example, heavier contributors could be presented as red (or some other color) while lesser contributors may be presented in other colors such as green, blue, gray, etc. In other embodiments, clinical risk factors may be presented as, for instance, pie charts. The relative contribution of each clinical risk factor may be presented visually as a relative-sized slice of the pie chart. In some embodiments, one or more tiles (or aspects thereof) may be animated to convey relative contributions of underlying clinical risk factors. For example, tiles associated with heavy contributors may blink, sparkle, etc., while tiles associated with lesser contributors may be static. In some embodiments, coloring (or other visual indication) of the indication of each clinical risk may be selected based on other parameters, such as when the status of the clinical risk factor was last assessed (e.g., stale versus fresh) and/or changed.

In some embodiments, GUI 232 of FIG. 2B (or GUI 230 of FIG. 2A) may be interactive. For example, “hovering” a mouse or other user-controllable element over a particular tile may cause additional information to be presented about the particular clinical risk factor, e.g., in a pop-up window. This additional information may include, for instance, information about when/how the clinical risk factor was learned/assessed, the clinical risk factor's actual assigned weight in an underlying health assessment model, etc. In some embodiments, a user may be able to select a tile to alter various aspects associated with the clinical risk factor. For example, a user could select a particular tile and be presented with an interface that is operable by the user to alter a weight or “theta” (e.g., by typing a new weight, sliding a slider along a weight scale, etc.) used in the underlying model. As another example, a user could select a tile to operate a similar user interface that allows the user to change the value for the clinical risk factor. GUI 232 could then visually update itself automatically, to inform the user how the change(s) impact the output and/or relative contributions of other clinical risk factors.

In various embodiments, a GUI such as GUI 232 in FIG. 2B may be presented in response to various events. For example, another GUI may be presented with one or more model-based health assessment scores associated with a patient. A user may select (e.g., click, hover over, etc.) a particular model-based health assessment score to be presented with GUI 232, at which point the user may be informed which clinical risk factors are at play, and how much each clinical risk factor contributed to the overall score.

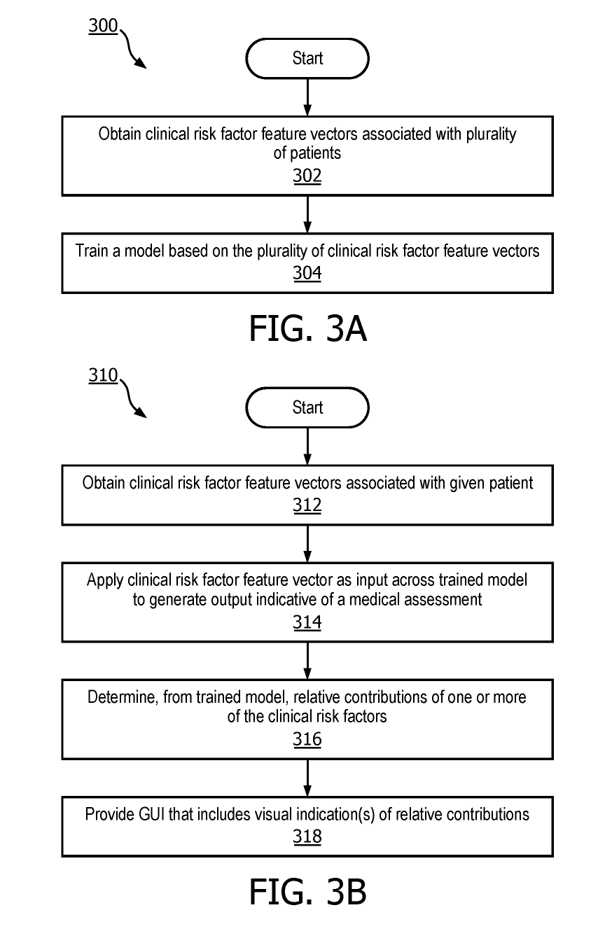

Referring now to FIG. 3A, an example method 300 of training a model (e.g., a machine learning classifier) is depicted. For the sakes of brevity and clarity, the operations of FIG. 3A and other flowcharts disclosed herein will be described as being performed by a system. However, it should be understood that one or more operations may be performed by different components of the same or different systems. For example, many of the operations may be performed by health assessment engine 102, e.g., to train one or more models of model database 106. And while FIG. 3A depicts supervised training of a model, this is not meant to be limiting. In various embodiments, various models to which techniques described herein are applicable may be trained using unsupervised techniques as well (e.g., by clustering various unlabeled data points/feature vectors).

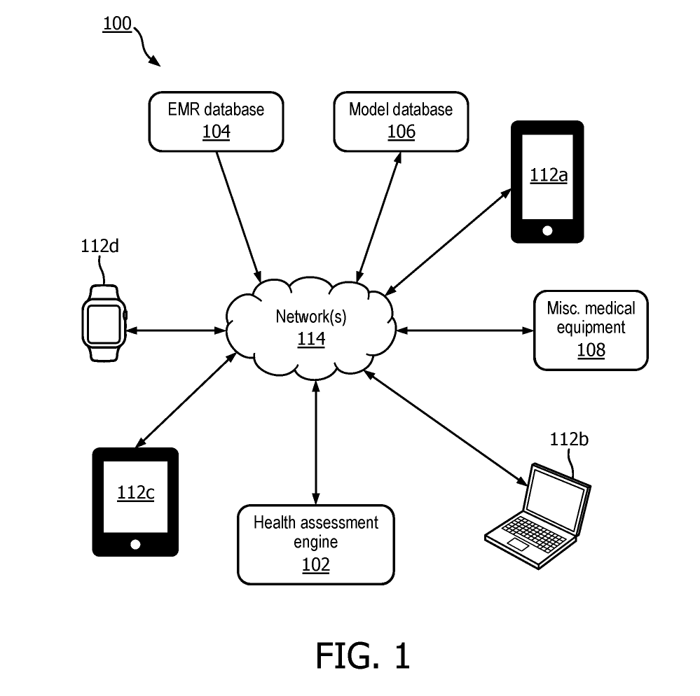

At block 302, the system may obtain a plurality of “labeled” clinical risk factor feature vectors associated with a plurality of patients, e.g., from EMR database 104 in FIG. 1. As noted above, these clinical risk factor feature vectors may include, as features, a wide variety of clinical risk factors associated with patients. These health indicator features may include but are not limited to age, gender, weight, blood pressure, temperature, pulse, central venous pressure (“CVP”), electrocardiogram (“EKG”) readings, oxygen levels, genetic indicators such as hereditary and/or racial indicators, chronic conditions (e.g., diabetes, obesity, chronic heart failure), test results, and so forth.

The labels associated with each clinical risk factor feature vector may come in various forms. In embodiments in which the model is trained to provide binary output (e.g., binary linear regression), the labels may be binary as well. For example, the label could represent a prior health assessment indicating that the patient has, or does not have, some chronic condition, or is (or is not) a suitable candidate for some type of medical intervention. In embodiments in which the model being trained to provide non-binary output (e.g., a score such as AKI), the labels may take the form of previously-calculated or otherwise assessed scores associated with each clinical risk factor feature vector.

At block 304, the system may train a model such as machine learning classifier (e.g., regression-based), a neural network model, a decision tree, etc., based on the plurality of clinical risk factor vectors obtained at block 302. In various embodiments, the model may be trained at block 304 so that subsequent input, such as subsequent unlabeled clinical risk factor feature vectors, may be applied across the model as input. Output may be generated in the form of classifications (e.g., binary or otherwise), scores, and so forth. Based on these training examples, an inferred function may be produced that can be used to map subsequent clinical risk factor vectors to various outputs.

The operations of block 304 may depend on the type of model used. For example, with logistic regression and other similar models, various optimization technique may be employed to, for instance, minimize a cost function. For example, various optimization methods, such as gradient descent, stochastic gradient descent, batch gradient descent, application of the normal equations, etc., may be applied to improve the accuracy of the model. With neural networks, techniques such as back propagation may be employed, e.g., in conjunction with gradient descent, to train the neural network.

In some embodiments, a model may be initiated, e.g., at a location such as a hospital or throughout a geographic area containing multiple medical facilities, e.g., in a preconfigured state (e.g., already trained with default training data). After initiation, a sliding temporal window (e.g., six months) of retrospective data may be used to update the model to more recent clinical risk factor data.

FIG. 3B schematically illustrates an example method 310 of applying clinical risk factors associated with a subsequent patient as input across one or more models. At block 312, the system may receive a plurality of clinical risk factors associated with a patient. For example, health assessment engine 102 may obtain a plurality of clinical risk factors associated with a particular patient from EMR database 104. At block 314, the system may apply the plurality of clinical risk factors as input across a trained model to generate output indicative of a medical assessment associated with the patient. This output may come in various forms, such as a classification (binary or otherwise), a score, etc. Scores can indicate various things, such as a likelihood of an adverse outcome from medical intervention, likelihood of developing a chronic condition, a particular health rating (e.g., patient acuity), etc.

At block 316 (which may occur before or after one or more operations of block 314), the system may determine, based on the trained model, relative contributions of one or more of the clinical risk factors to the output of the trained model. In some embodiments, the relative contributions may be determined based on, for instance, relative weights associated with each risk factor. For example, coefficients of regression models may be indicative of relative weights associated with each input clinical risk factor.

With neural networks, the clinical risk factors may be applied as input. The neural network may include any number of hidden layers of neurons. Each neuron (or in some cases, each edge between neurons) may include a corresponding weight. In instances in which edges between neurons have weights, those weights may or may not be static, and the neurons themselves may have associated “activities” or “energies.” Weights associated with neurons (or edges between neurons) relatively close to the input (as opposed to the final level(s) of neurons closer to the output) may be used as indicators of weights that may affect contribution of one or more clinical risk factors to an output. Additionally or alternatively, relative contributions of inputs to a neural network may be determined using other techniques, such as sensitivity analysis, the so-called “Lek profile method,” fuzzy curves, mean square error, partial derivative method, sum of squares error (“SSE”), deconstruction of model weights, etc.

In other embodiments, semantic meanings of late-level neurons (e.g., the last row of neurons prior to the output) may be determined and interpreted as relative contributions. For example, suppose a particular neural network is trained to provide, as output, one health assessment classification from an enumerated list of potential classifications (similar to convolutional neural network's estimating between different subjects as being depicted in an input image). Each last-level neuron may be associated with one of the enumerated choices. The ultimate output of the neural network model may be the most likely health assessment classification in view of the input clinical risk factors. However, a GUI generated using techniques described herein may present a GUI similar to that depicted in FIG. 2B which portrays each possibility and its associated likelihood. Accordingly, a clinician can see the best guess (indicated by the output of the neural network model) as well as the second best guess, the third best guess, and so forth. Additionally or alternatively, in some embodiments, early-level neurons may be closely related to input clinical risk factors (e.g., congestive heart failure, and late-level neurons may be closely related to high level medical concepts (e.g., cardiac disease).

At block 318, the system may provide, e.g., on a display, a graphical user interface (e.g., GUI 232 of FIG. 2B) that includes visual indication(s) of the relative contribution(s). For example, in FIG. 2B, the visual indication took the form of a height of each tile along the z-axis (i.e., out of the page). Referring back to FIG. 1, various client devices 112a-d may be used to view such a graphical user interface.

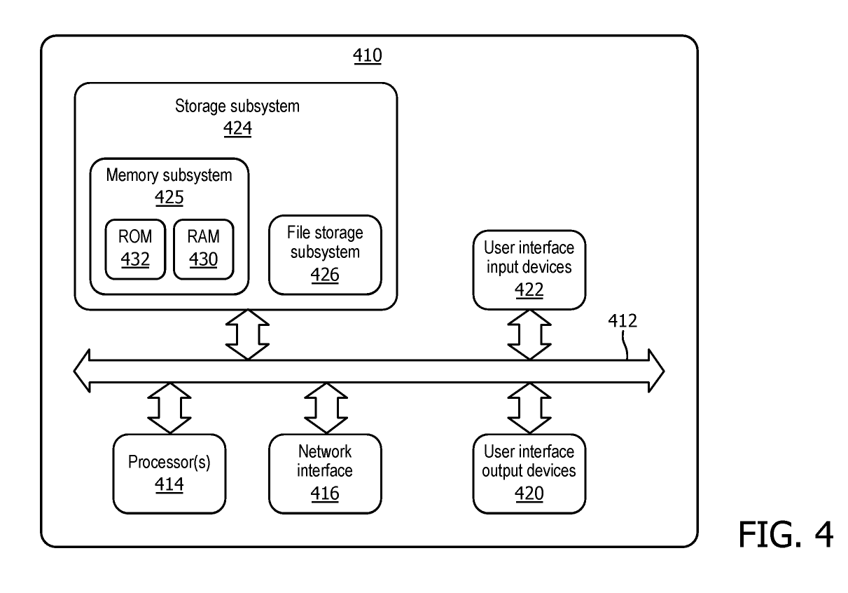

FIG. 4 is a block diagram of an example computer system 410. Computer system 410 typically includes at least one processor 414 which communicates with a number of peripheral devices via bus subsystem 412. These peripheral devices may include a storage subsystem 424, including, for example, a memory subsystem 425 and a file storage subsystem 426, user interface output devices 420, user interface input devices 422, and a network interface subsystem 416. The input and output devices allow user interaction with computer system 410. Network interface subsystem 416 provides an interface to outside networks and is coupled to corresponding interface devices in other computer systems.

User interface input devices 422 may include a keyboard, pointing devices such as a mouse, trackball, touchpad, or graphics tablet, a scanner, a touchscreen incorporated into the display, audio input devices such as voice recognition systems, microphones, and/or other types of input devices. In general, use of the term “input device” is intended to include all possible types of devices and ways to input information into computer system 410 or onto a communication network.

User interface output devices 420 may include a display subsystem, a printer, a fax machine, or non-visual displays such as audio output devices. The display subsystem may include a cathode ray tube (CRT), a flat-panel device such as a liquid crystal display (LCD), a projection device, or some other mechanism for creating a visible image. The display subsystem may also provide non-visual display such as via audio output devices. In general, use of the term “output device” is intended to include all possible types of devices and ways to output information from computer system 410 to the user or to another machine or computer system.

Storage subsystem 424 stores programming and data constructs that provide the functionality of some or all of the modules described herein. For example, the storage subsystem 424 may include the logic to perform selected aspects of methods 300 and/or 310, and/or to implement one or more of health assessment engine 102, EMR database 104, model database 106, and/or any of client devices 112a-d.

These software modules are generally executed by processor 414 alone or in combination with other processors. Memory 425 used in the storage subsystem can include a number of memories including a main random access memory (RAM) 430 for storage of instructions and data during program execution and a read only memory (ROM) 432 in which fixed instructions are stored. A file storage subsystem 426 can provide persistent storage for program and data files, and may include a hard disk drive, a floppy disk drive along with associated removable media, a CD-ROM drive, an optical drive, or removable media cartridges. The modules implementing the functionality of certain implementations may be stored by file storage subsystem 426 in the storage subsystem 424, or in other machines accessible by the processor(s) 414.

Bus subsystem 412 provides a mechanism for letting the various components and subsystems of computer system 410 communicate with each other as intended. Although bus subsystem 412 is shown schematically as a single bus, alternative implementations of the bus subsystem may use multiple busses.

Computer system 410 can be of varying types including a workstation, server, computing cluster, blade server, server farm, or any other data processing system or computing device. Due to the ever-changing nature of computers and networks, the description of computer system 410 depicted in FIG. 4 is intended only as a specific example for purposes of illustrating some implementations. Many other configurations of computer system 410 are possible having more or fewer components than the computer system depicted in FIG. 4.

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 1111.03. It should be understood that certain expressions and reference signs used in the claims pursuant to Rule 6.2(b) of the Patent Cooperation Treaty (“PCT”) do not limit the scope

What is claimed is:

1. A computer-implemented method comprising:
receiving a plurality of clinical risk factors associated with a patient;
applying the plurality of clinical risk factors as input across a trained model to generate a score associated with the patient;
determining, based on the trained model, a first contribution of a first clinical risk factor of the plurality of clinical risk factors to the score;
determining, based on the trained model, a second contribution of a second clinical risk factor of the plurality of clinical risk factors to the score; and
providing, on a display, a graphical user interface that includes at least a first visual indication of the first contribution and a second visual indication of the second contribution.

2. The method of claim 1, wherein the first and second visual indications comprise first and second blocks having first and second heights that are selected based on the first and second contributions, respectively.

3. The method of claim 2, wherein the display is a two-dimensional display having an x-axis and a y-axis, and the first and second blocks are rendered three-dimensionally so that the first and second blocks extend along a simulated z-axis by the first and second heights.

4. The method of claim 1, wherein the first and second contributions are determined based on first and second weights obtained from the trained model.

5. The method of claim 1, wherein the trained model comprises a logistic regression model.

6. The method of claim 1, wherein the trained model comprises a neural network.

7. The method of claim 6, wherein the first and second contributions are determined based on top level neurons of the neural network.

8. The method of claim 1, wherein the first and second visual indications comprise one or more font attributes used to display, in the graphical user interface, text associated with the first and second clinical risk factors, respectively.

9. A system comprising one or more processors and memory operably coupled with the one or more processors, wherein the memory stores instructions that, in response to execution of the instructions by one or more processors, cause the one or more processors to:
receive a plurality of clinical risk factors associated with a patient;
apply the plurality of clinical risk factors as input across a trained model to generate output associated with the patient;
determine, based on the trained model, a first contribution of a first clinical risk factor of the plurality of clinical risk factors to the output;
determine, based on the trained model, a second contribution of a second clinical risk factor of the plurality of clinical risk factors to the output; and
provide, on a display, a graphical user interface that includes at least a first visual indication of the first contribution and a second visual indication of the second contribution.

10. The system of claim 9, wherein the first and second visual indications comprise first and second blocks having first and second heights that are selected based on the first and second contributions, respectively.

11. The system of claim 10, wherein the display is a two-dimensional display having an x-axis and a y-axis, and the first and second blocks are rendered three-dimensionally so that the first and second blocks extend along a simulated z-axis by the first and second heights.

12. The system of claim 9, wherein the first and second contributions are determined based on first and second weights obtained from the trained model.

13. The system of claim 9, wherein the trained model comprises a logistic regression model.

14. The system of claim 9, wherein the trained model comprises a neural network.

15. At least one non-transitory computer-readable medium comprising instructions that, in response to execution of the instructions by one or more processors, cause the one or more processors to perform the following operations:
receiving a plurality of clinical risk factors associated with a patient;
applying the plurality of clinical risk factors as input across a trained model to generate a output associated with the patient;
determining, based on the trained model, a first contribution of a first clinical risk factor of the plurality of clinical risk factors to the output;
determining, based on the trained model, a second contribution of a second clinical risk factor of the plurality of clinical risk factors to the output; and
providing, on a display, a graphical user interface that includes at least a first visual indication of the first contribution and a second visual indication of the second contribution.
